(19) United States
(12) Patent Application Publication
(10) Pub. No.: US 2013/0171660 A1
(43) Pub. Date: Jul. 4, 2013

(54) BLOOD MARKER FOR RENAL CANCER

(71) Applicants: SHIMADZU CORPORATION, Kyoto-shi (JP); EDUCATIONAL CORPORATION MUKOGAWA GAKUIN, Nishinomiya-shi (JP); HYOGO COLLEGE OF MEDICINE, Nishinomiya-shi (JP)

(72) Inventors: Eiichi Matsuo, Kyoto-shi (JP); Naoki Kaneko, Kyoto-shi (JP); Makoto Watanabe, Kyoto-shi (JP); Osamu Nishimura, Kyoto-shi (JP); Noboru Okamura, Nishinomiya-shi (JP); Akinobu Gotoh, Nishinomiya-shi (JP)

(73) Assignees: SHIMADZU CORPORATION, Kyoto-shi (JP); HYOGO COLLEGE OF MEDICINE, Nishinomiya-shi (JP); EDUCATIONAL CORPORATION MUKOGAWA GAKUIN, Nishinomiya-shi (JP)

(21) Appl. No.: 13/728,932

(22) Filed: Dec. 27, 2012

(30) Foreign Application Priority Data
Dec. 28, 2011 (JP) 2011-289649

(51) Int. Cl. G01N 33/68; C12N 9/96

(52) U.S. Cl. CPC G01N 33/6893; C12N 9/96
USPC 435/7.4; 530/396; 435/232; 435/7.92; 435/188

(57) ABSTRACT

The present invention provides a blood marker for renal cancer, more specifically, a blood marker that can be practically used for clinical diagnosis of renal cancer. The present invention also provides a blood marker that can be practically used for follow-up after treatment such as surgery and during treatment such as medication for renal cancer. A blood marker for renal cancer selected from the group consisting of Galectin-1, Galectin-3, and α-enolase. Galectin-1 and/or Galectin-3 as a blood marker for renal cancer for use in an examination performed before diagnostic imaging. α-Enolase as a blood marker for renal cancer for use in monitoring during and/or after treatment for renal cancer.

### BACKGROUND OF THE INVENTION

1. Field of the Invention

The present invention relates to a technique for clinical diagnosis, examination, and follow-up. Particularly, the present invention relates to a blood marker for renal cancer.

2. Disclosure of the Related Art

Early detection of cancer is important to reduce a cancer death rate. However, clinical diagnosis of renal cancer is mostly performed by diagnostic imaging such as CT or MRI. More specifically, renal cancer is often accidentally detected by a thorough medical checkup or an examination for another disease, or by subjective symptoms such as hematuria or lumbar backache. Most of patients diagnosed as having cancer in this way are already in advanced stages.

Effective treatment for renal cancer is surgical resection. As treatment for metastatic renal cancer, immune therapy using interferon a or interleukin-2 is also used. However, according to the Journal of Urology, 2005, 174 (2): 466-472 (Non-Patent Document 1), 27.6% of patients with localized cancer relapse within 5 years after surgery. The relapse rate among patients with locally-advanced cancer reaches 64%. The response rate of immune therapy is as low as about 11 to 26%.

According to WO2008/032868 (Patent Document 1), many proteins including Galectin 1 have been found as renal cancer-associated proteins whose expression in renal cancer tissue increases or decreases by proteomic analysis of cancerous and non-cancerous parts of renal tissues surgically extracted from patients with renal cancer.

According to Proteomics 2008, 8(15): 3194-3203 (Non-Patent Document 2), many proteins including Galectin 1, Galectin 3, and α-enolase have been found as renal cancer-associated proteins whose expression in renal cancer tissue increases or decreases by proteomic analysis of cancerous and non-cancerous parts of renal tissues surgically extracted from patients with renal cancer.

According to WO2007/142347 (Patent Document 2), many proteins including Galectin 1 have been found as colon cancer-associated proteins whose expression in colon cancer tissue increases or decreases by proteomic analysis of cancerous and non-cancerous parts of tissues surgically extracted from patients with colon cancer.

According to Oncology Reports, 2011, 25, 1217-1226 (Non-Patent Document 3), Galectin-1, Galectin-3, and Galectin-4 have been found as colon cancer-associated proteins whose blood concentrations in patients with colon cancer are significantly high.
Patent Document 1: WO2008/032868
Patent Document 2: WO2007/142347

Non-Patent Document 1: Lam J S, Shvarts O, Leppert J T, Pantuck A J, Figlin R A, and Belldegrun A S; Postoperative surveillance protocol for patients with localized and locally advanced renal cell carcinoma based on a validated prognostic nomogram and risk group stratification system. The Journal of Urology, 2005, Vol. 174, No. 2, pp. 466-472

Non-Patent Document 2: Okamura N, Masuda T, Gotoh A, Shirakawa T, Terao S, kaneko N, Suganuma K, Watanabe M, Matsubara T, Seto R, Matsumoto J, Kawakami M, Yamamori M, Nakamura T, Yagami T, Sakaeda T, Fujisawa M, Nishimura O, and Okumura K; Quantitative proteomic analysis to discover potential diagnostic markers and therapeutic targets in human renal cell carcinoma. Proteomics 2008, Vol. 8, No. 15, pp. 3194-3203

Non-Patent Document 3: Watanabe M, Takemasa I, Kaneko N, Yokoyama Y, Matsuo E, Iwasa S, Mori M, Matsuura N, Monden M, and Nishimura O; Clinical significance of circulating galectins as colorectal cancer markers. Oncology Reports, 2011, Vol. 25, pp. 1217-1226

### SUMMARY OF THE INVENTION

A blood test is a simple, relatively cheap, and less invasive detection method. However, there are no clinically available blood markers for renal cancer, and therefore development of such markers is needed. Further, in view of the high relapse rate of renal cancer after surgery, it is also important to quickly detect relapse after surgery. It is therefore an object of the present invention to provide a blood marker for renal cancer. More specifically, the object of the present invention is to provide a blood marker that can be practically used for clinical diagnosis of renal cancer, that is, for cancer detection.

Further, in consideration of the fact that immune therapy is effective although its response rate is low and molecularly targeted drugs have recently started to be used, it is also important to monitor the effects of such immune therapy or chemotherapy. Further, as described above, follow-up after surgery is also important. It is therefore another object of the present invention to provide a blood marker that can be practically used for follow-up during and after treatment for renal cancer.

The present inventors have intensively studied, and as a result, have found that the above objects of the present invention can be achieved by a protein selected from the group consisting of Galectin-1, Galectin-3, and α-enolase. This finding has led to the completion of the present invention.

The present invention includes the following inventions.

(1) A blood marker for renal cancer selected from the group consisting of Galectin-1, Galectin-3, and α-enolase.
(2) Galectin-1 as a blood marker for renal cancer for use in an examination performed before diagnostic imaging.
(3) Galectin-3 as a blood marker for renal cancer for use in an examination performed before diagnostic imaging.
(4) A combination of Galectin-1 and Galectin-3 as a blood marker for renal cancer for use in an examination performed before diagnostic imaging.
(5) α-Enolase as a blood marker for renal cancer for use in an examination performed before diagnostic imaging.
(6) α-Enolase as a blood marker for renal cancer for use in monitoring during and/or after treatment for renal cancer.
(7) A renal cancer marker detection kit comprising an enzyme-labeled α-enolase antibody.
(8) A method for analyzing a renal cancer marker in a collected blood sample, the method comprising the step of acquiring a level of a renal cancer marker selected from the group consisting of Galectin-1, Galectin-3, and α-enolase in a collected blood sample derived from an individual, and evaluating the acquired marker level based on whether said acquired marker level is higher or lower than a reference level of the renal cancer marker.

The “marker level” in the above (8) basically represents a concentration of a marker, but the “level” may be a measure other than concentration used by those skilled in the art.

The present invention provides a blood marker for renal cancer. This makes it possible to improve the early detection rate of renal cancer by a blood test that is a simple and relatively cheap method. Further, the present invention makes it possible to achieve a method capable of easily detecting renal cancer, and a method for monitoring the effects of treatment for renal cancer during and after the treatment. That is, the relapse of cancer after surgery and the effects of non-surgical treatment such as immune therapy or drug therapy can be monitored by a simple blood test.

### BRIEF DESCRIPTION OF THE DRAWINGS

FIG. 1 shows box plots representing the distribution of concentrations of Galectin-1 (FIG. 1A), Galectin-3 (FIG. 1B), α-enolase (FIG. 1C), and Calnexin (FIG. 1D) in blood samples collected from a group of healthy individuals (Control) and a group of patients with renal cancer (RCC).

FIG. 2 shows box plots representing the distribution of concentrations of CNDP dipeptidase 2 (FIG. 2E), Lectin mannose-binding 2 (FIG. 2F), Triosephosphate isomerase (FIG. 2G), and MHC class I antigen A (FIG. 2H) in the blood samples collected from the group of healthy individuals (Control) and the group of patients with renal cancer (RCC).

FIG. 3 shows ROC curves comparing discrimination between patients with renal cancer and healthy individuals based on the concentrations of Galectin-1, Galectin-3, and α-enolase in the collected blood samples. The vertical axis represents a positive rate (sensitivity (%)) and the horizontal axis represents a false-positive rate (100−specificity (%)).

FIG. 4 shows scatter diagrams of concentrations of Galectin-1 and Galectin-3 (FIG. 4A), concentrations of Galectin-1 and α-enolase (FIG. 4B), and concentrations of Galectin-3 and α-enolase (FIG. 4C) in the collected blood samples of the healthy individuals and the patients with renal cancer. White dots represent the patients with renal cancer (RCC) and black dots represent the healthy individuals (control).

FIG. 5 is a graph representing changes in the concentration of α-enolase in blood samples of respective patients who have undergone surgical resection of renal cancer, collected before the surgery, 4 weeks after the surgery, and 12 weeks after the surgery.

### DETAILED DESCRIPTION OF THE INVENTION

### [1. Renal Cancer Marker]

The present invention provides Galectin-1, Galectin-3, and α-enolase as renal cancer markers. These markers are proteins whose concentrations in a collected blood sample are significantly different between a group of patients with renal cancer and a group of healthy individuals. The proteins according to the present invention are highly reliable as markers because they were discovered by examining a group of patients with renal cancer and a group of healthy individuals similar in male-to-female ratio and age. These proteins are present in significantly high concentrations in collected blood samples of patients with renal cancer.

All the markers according to the present invention can be used in a blood test. The blood test is preferably performed as a simple examination (e.g., as a preliminary diagnosis or a primary diagnosis, or as one of examinations performed for a medical checkup or a thorough medical checkup) before diagnosis is confirmed by diagnostic imaging. Among the markers according to the present invention, Galectin-1 and Galectin-3 are particularly useful for such an examination performed before diagnostic imaging.

Further, Galectin-1 and Galectin-3 are preferably used in combination in such an examination performed before diagnostic imaging. A combination (intersection) of markers inevitably causes a reduction in sensitivity, but a combination of Galectin-1 and Galectin-3 shows a smaller reduction in sensitivity and has high specificity.

On the other hand, α-enolase is particularly useful for monitoring after treatment for renal cancer.

### [2. Collected Blood Sample]

The renal cancer marker according to the present invention can be detected and analyzed in a collected blood sample. Therefore, the level of the renal cancer marker in a collected blood sample is analyzed by a method according to the present invention.

A collected blood sample is a sample directly subjected to the step of acquiring the level of the renal cancer marker, and includes whole blood, blood plasma, blood serum, and the like. The collected blood sample can be prepared by appropriately treating whole blood collected from an individual. Treatment performed to prepare a blood sample from collected whole blood is not particularly limited, and any treatment may be performed as long as it is clinically acceptable. For example, centrifugal separation may be performed. Further, the collected blood sample subjected to the measurement step (i.e., the acquiring step) may be one that has been stored appropriately at low temperatures such as by freezing in the course of or after the step of its preparation. It is to be noted that in the present invention, the collected blood sample is discarded without being returned to the individual who was the source.

Examples of an individual who will be the source of the collected blood sample include patients who undergo a medical checkup or a thorough medical checkup, patients with a chief complaint of subjective symptoms such as hematuria, lumbar backache or the like, and renal cancer patients who have received treatment for renal cancer and are required to be monitored. Examples of treatment for renal cancer include surgery and non-surgical treatment. Examples of non-surgical treatment include immune therapy and drug therapy.

### [3. Analysis of Level of Renal Cancer Marker in Collected Blood Sample]

According to the present invention, the level of the renal cancer marker in a collected blood sample is analyzed by a comparison between measured level and reference level of the renal cancer marker. For a more accurate analysis, both of the measured level and the reference level to be compared are preferably based on blood samples prepared under the same conditions (e.g., pretreatment conditions, storage conditions).

The method of the present invention comprises the step of measuring the level of the renal cancer marker in a collected blood sample derived from blood to acquire a measured level of the renal cancer marker, and then comparing the measured level of the renal cancer marker with a reference level of the renal cancer marker.

### [3-1. Reference Level]

The reference level is used as a criterion for determining the clinical state of renal cancer. As described above, the renal cancer marker of the present invention shows a significant difference in its concentration in a collected blood sample between a group of patients with renal cancer and a group of healthy individuals. Therefore, the setting of appropriate reference level of the renal cancer marker is effective for discrimination between these groups.

A specific example of the reference level is usually a threshold value specific to each of the renal cancer markers. The threshold value used in the present invention can be previously set for each kind of renal cancer marker protein depending on race, age, etc. The threshold value can be set by reference to respective measured levels of the renal cancer marker of a healthy individual group and a renal cancer patient group acquired by measuring the amounts of the renal cancer marker present in respective collected blood samples derived from individuals belonging to the healthy individual group and individuals belonging to the renal cancer patient group by a measurement method that will be described later.

The threshold value (cut-off value) may be selected so as to achieve high diagnostic accuracy. The threshold value may vary depending on the kind of marker protein used, but can be appropriately selected by those skilled in the art from among, for example, cut-off values that yield a specificity of 60% or higher, preferably 80% or higher, more preferably 90% or higher and a sensitivity of 30% or higher, preferably 50% or higher, more preferably 60% or higher.

A method for setting the threshold value is appropriately selected by those skilled in the art. One example of the method is an ROC Curve (Receiver Operating Characteristic Curve) analysis.

As another specific example of the reference level, a measured level of the renal cancer marker in a blood sample previously collected from the same individual is also acceptable.

### [3-2. Use of Marker for Examination Before Diagnostic Imaging]

When the protein according to the present invention (particularly preferably, Galectin-1 and/or Galectin-3) is used as a marker for examination before diagnostic imaging, a reference level of said marker is used as a criterion for discrimination between collected blood samples derived from renal cancer patients and collected blood samples derived from healthy individuals. More specifically, the reference level of a marker for examination before diagnostic imaging is a threshold value of the marker for examination before diagnostic imaging.

When a measured level of the marker in a collected blood sample is higher than the reference level, it is possible to judge that there is a high possibility that the individual who is the source of the blood sample has renal cancer (i.e., the individual is strongly suspected of having renal cancer). On the other hand, when a measured level of the marker in a collected blood sample is lower than the reference level, it is possible to judge that there is a high possibility that the individual who is the source of the blood sample is healthy (i.e, the individual has a low probability of having renal cancer). In the former case, diagnostic imaging may be further performed for confirmation of renal cancer.

[3-3. Use of Marker for Monitoring During and after Treatment for Renal Cancer]

When the protein according to the present invention (more specifically, α-enolase) is used as a marker for monitoring during and/or after treatment for renal cancer, a reference level of said marker is used as a criterion for discrimination between collected blood samples derived from renal cancer patients and collected blood samples derived from patients who have received effective treatment for renal cancer. More specifically, as in the case of the above-described marker for examination before diagnostic imaging, a threshold value is used as the reference level.

An individual who is monitored during and/or after treatment for renal cancer using the marker according to the present invention is one whose marker level in blood before treatment was higher than the reference level. Specific examples of the treatment for renal cancer include surgery and medication. When the marker according to the present invention is used for monitoring during treatment, the treatment is preferably non-surgical treatment, more specifically medication. When the marker according to the present invention is used for monitoring after treatment, the treatment is preferably surgical treatment, more specifically surgery.

During treatment for renal cancer or at some point in time during treatment for renal cancer, a collected blood sample is subjected to measurement of α-enolase to acquire a measured level. When the measured level is lower than the reference level, it is possible to judge that the treatment for renal cancer was sufficiently effective for the individual who was the source of the collected blood sample at that time (or cancer has disappeared). On the other hand, when the measured level is higher than the reference level, it is possible to judge that the treatment for renal cancer was not sufficiently effective for the individual who was the source of the collected blood sample at that time (or cancer has not disappeared).

The method according to the present invention makes it possible to make an examination after surgery to perform follow-up to detect relapse of renal cancer. It is to be noted that as the follow-up for detection of relapse of renal cancer after surgery, only the above-described follow-up may be performed without any particular treatment, or follow-up combined with non-surgical treatment may be performed.

Further, the method according to the present invention makes it possible to make an examination during non-surgical treatment to follow-up the effects of treatment for renal cancer. The follow-up of effects of treatment for renal cancer during non-surgical treatment also makes it possible to make a judgment as to whether the treatment needs to continue or not. For example, when the measured level of α-enolase in a collected blood sample at some point in time during treatment is below the threshold value, the treatment may be terminated, and when the measured level of α-enolase in a collected blood sample at said point in time is still higher than the threshold value, non-surgical treatment may be further repeated.

It is to be noted that even when the measured level of α-enolase at some point in time after treatment is below the threshold value, if the measured value of α-enolase exceeds the threshold value in a subsequent examination, it is possible to judge that there is suspicion of relapse or metastasis of renal cancer. The method according to the present invention can be easily implemented by a blood test, and therefore the level of α-enolase can be repeatedly measured even after the completion of treatment, which contributes to early detection of relapse or metastasis of cancer.

### [3-4. Measurement Method]

The measurement of the renal cancer marker according to the present invention is preferably performed by a test based on biospecific affinity. The test based on biospecific affinity is a method well known to those skilled in the art and is not particularly limited, but is preferably an immunoassay. Specific examples of the immunoassay include competitive and non-competitive assays such as western blotting, radioimmunoassay, ELISA (Enzyme-Linked ImmunoSorbent Assay) (including sandwich immunoassay, competitive assay, and direct binding assay), immunoprecipitation, precipitation reaction, immunodiffusion, immunoagglutination measurement, complement binding reaction analysis, immunoradiometric assay, fluorescence immunoassay, and protein A immunoassay. In the immunoassay, the renal cancer marker in a collected blood sample is detected by an antibody that binds to the said renal cancer marker.

The antibody that binds to the renal cancer marker can be appropriately determined by those skilled in the art. For example, a renal cancer marker protein antibody (a monoclonal antibody or polyclonal antibody) or a labeled renal cancer marker protein antibody is used. A label in the labeled marker protein antibody may be a fluorescent compound and/or an enzyme protein. As the fluorescent compound and the enzyme protein, those acceptable in a measurement system using an antibody are appropriately selected by those skilled in the art. For example, the enzyme protein may be selected from the group consisting of peroxidase, alkaline phosphatase, and β-galactosidase.

A specific protocol for preparation and labeling of an antibody against the renal cancer marker protein can be easily selected by those skilled in the art.

The measurement of the renal cancer marker is performed by bringing a collected blood sample into contact with an antibody under the condition that the renal cancer marker protein to be measured and the antibody against the renal cancer marker protein can form an immunocomplex.

A more specific protocol for immunoassay can be easily selected by those skilled in the art.

One example of the protocol is as follows. A capture antibody is, for example, adsorbed onto a substrate or a well inner wall to obtain a solid phase-capture antibody. As the capture antibody, a polyclonal antibody or a monoclonal antibody against the marker protein, which recognizes an epitope different from that recognized by the above-described marker protein antibody or labeled marker protein antibody, is preferably used. The concentration of a capture antibody solution used to obtain a solid phase capture antibody is appropriately determined by those skilled in the art using the protocol, and may be, for example, in the range of 0.1 to 20 μg/mL, preferably 1 to 10 μg/mL. One example of the concentration may be 5 μg/mL.

A collected blood sample is added to the solid phase-capture antibody under the condition that the capture antibody and the marker protein in the blood sample can form an immunocomplex. If necessary, the blood sample may be previously diluted appropriately. The dilution factor of the blood sample is appropriately determined by those skilled in the art using the protocol. The upper limit of the dilution factor is not particularly limited, but may be, for example, in the range of about 1- to 100-fold. One example of the dilution factor may be 10-fold.

The substrate or well is washed, and then the above-described labeled marker protein antibody is added thereto under the condition that the marker protein derived from the blood sample and bound to the capture antibody, and the labeled marker protein antibody can form an immunocomplex. The concentration of the labeled marker protein antibody to be added is appropriately determined by those skilled in the art, and may be, for example, in the range of 0.05 to 5 μg/mL, preferably 0.1 to 2 μg/mL. One example of the concentration may be 0.5 μg/mL.

Then, the substrate or well is washed, and a signal derived from the labeled marker protein antibody bound to the marker protein is detected. For example, when the antibody is labeled with a fluorescent compound, the amount of fluorescence derived from the label can be measured. Further, when the antibody is labeled with an enzyme protein, a signal can be measured by adding a substrate for the enzyme protein and detecting chemiluminescence derived from a compound obtained by decomposition of the substrate.

Alternatively, after the substrate or well is washed, a non-labeled marker protein antibody and then a labeled secondary antibody may be added to detect a signal in the same manner as described above. In this case, the non-labeled marker protein antibody is specifically recognized by the labeled secondary antibody, and therefore the marker protein can be specifically detected in an indirect manner.

[4. Kit for Detecting Renal Cancer Marker in Collected Blood Sample]

The present invention provides a renal cancer marker detection kit comprising a labeled α-enolase antibody. The labeled α-enolase antibody is an α-enolase antibody labeled with a substance selected from the group consisting of fluorescent compound and enzyme. Examples of the enzyme include peroxidase, alkaline phosphatase, β-galactosidase, and the like. The renal cancer marker detection kit according to the present invention can be used for the above-described renal cancer marker analysis.

The labeled α-enolase antibody may be provided as a solution prepared to have the above-described concentration.

The renal cancer marker detection kit may include, as an additional item, the above-described capture antibody selected from the group consisting of polyclonal anti-α-enolase antibody and monoclonal anti-α-enolase antibody. The capture antibody may be provided as a solution prepared to have the above-described concentration or as a solid phase on the surface of a substrate or on the inner wall of a well.

### EXAMPLES

Hereinbelow, the present invention will be described more specifically with reference to examples, but is not limited to the following examples.

### Example 1

### Construction of ELISA System

First, ELISA measurement systems were prepared for eight kinds of proteins whose concentrations are known to increase in renal cancer tissue [α-enolase, Calnexin (for reference), CNDP dipeptidase 2 (for reference), Galectin-1, Galectin-3, Lectin mannose-binding 2 (for reference), Triosephosphate isomerase (for reference), and MHC class I antigen A (for reference)].

Among them, the measurement systems for Galectin-1 and Galectin-3 were constructed using a capture antibody, a detection antibody, and a detection reagent shown in Table 1. The detection antibody used was labeled with the labeling protein (i.e., a protein for labeling) shown in Table 1.

A capture antibody solution (5 μg/mL) was added to each of the wells of a 96-well plate (manufactured by Maxisorp) to obtain a solid-phase antibody. The solid-phase capture antibody was obtained using IMMUNO-TEK ELISA Construction System (ZeptoMetrix, Buffalo, N.Y.).

TABLE 1

| | Galectin-1 ELISA | Galectin-3 ELISA |
|---|---|---|
| Recombinant | Abnova, Taiwan | R&D systems, |
| protein | | Minneapolis, MN |
| manufacturer | | |
| Capture antibody | Polyclonal anti-galectin-1 | Polyclonal anti-galectin-3 |
| (manufacturer) | antibody (R&D systems) | antibody (R&D systems) |
| Concentration of | 5 μg/mL | 5 μg/mL |
| Capture antibody | | |
| Detection antibody | Monoclonal anti- | Monoclonal anti- |
| (manufacturer) | galectin-1 antibody | galectin-3 antibody |
| | (Abnova) | (R&D systems) |
| Concentration | 0.5 μg/mL | 0.1 μg/mL |
| of labeled detection | | |
| antibody | | |
| Labeling protein | Alkaline Phosphatase | Peroxidase |
| (kit, manufacturer) | (Alkaline Phosphatase | (Peroxidase Labeling |
| | Labeling Kit-NH2, | Kit-SH, Dojindo |
| | Dojindo Molecular | Molecular |
| | Technologies, Inc.) | Technologies, Inc.) |
| Dilution factor of | 10-fold | 10-fold |
| Measurement | | |
| sample | | |
| Detection reagent | AP-Blue SpectraFX | TMB Two Component |
| (manufacturer) | Microwell and/or | HRP Microwell Substrate |
| | Membrane Substrate | (BioFX Laboratories, |
| | (BioFX Laboratories, | Inc., Owings Mills) |
| | Inc., Owings Mills) | |

The measurement systems for the other proteins were constructed using a recombinant protein, a capture antibody, a detection antibody, and an enzyme-labeled secondary antibody shown in Table 2. It is to be noted that the detection antibody used for detecting α-enolase was labeled with peroxidase enzyme.

TABLE 2

| | | Capture antibody | Detection antibody | Enzyme-labeled secondary |
|---|---|---|---|---|
| | Recombinant protein | (manufacturer) | (manufacturer) | antibody (manufacturer) |
| Protein | (manufacturer) | concentration | concentration | dilute concentration |
| α-enolase | Recombinant ENO1 | Mouse anti-human | Rabbit anti-human | — |
| | (Abnova, Taiwan) | α-enolase mAb | α-enolase pAb | |
| | | (Abnova) | (Proteintech Group, | |
| | | 3 μg/mL | Chicago, IL) | |
| | | | 0.2 μg/mL | |
| Calnexin | Recombinant Calnexin | Rabbit anti-human | Mouse anti-human | Anti-Mouse IgG HRP |
| | (assay designs, Ann | Calnexin pAb | Calnexin mAb | (Invitrogen, Carlsbad, |
| | Arbor, MI) | (GenScript USA Inc, | (BD, Franklin Lakes, NJ) | CA) |
| | | Piscataway, NJ) | 4 μg/mL | 1/10000 |
| | | 5 μg/mL | | |
| CNDP dipeptidase 2 | Recombinant CNDP2 | Rabbit anti-human | Sheep anti-human | Anti-Sheep IgG HRP |
| | (R&D Systems, | CNDP2 pAb | CNDP2 pAb | (Invitrogen) |
| | Minneapolis, MN) | 5 μg/mL | (R&D Systems) | 1/50000 |
| | | | 0.5 μg/mL | |
| Lectin mannose-binding 2 | Recombinant LMAN2 | Rabbit anti-human | Mouse anti-human | Anti-Mouse IgG HRP |
| | (Abnova) | LMAN2 pAb | LMAN2 mAb | (Invitrogen) |
| | | (Aviva Systems Biology) | (Abcam, Cambridge, UK) | 1/10000 |
| | | 5 μg/mL | 1 μg/mL | |
| MHC class I antigen A | Recombinant HLA-A | Rabbit anti-human | Goat anti-human | Anti-Goat IgG HRP |
| | (Abnova) | HLA-A pAb | HLA-A pAb | (Invitrogen) |
| | | (Proteintech Group) | (Santa cruz biotechnology, | 1/50000 |
| | | 5 μg/mL | Santa cruz, CA) | |
| | | | 0.5 μg/mL | |
| Triosephosphate isomerase | Recombinant TPI1 | Mouse anti-human | Rabbit anti-human | Anti-Rabbit IgG HRP |
| | (Abnova) | TPI1 mAb | TPI1 pAb | (Invitrogen) |
| | | (Abnova) | (Proteintech Group) | 1/50000 |
| | | 3 μg/mL | 0.1 μg/mL | |

The measuring range for calibration curve, detection limit, dilution factor of sample measured, and recovery rate based on spiked recovery test of each of the constructed ELISA measurement systems are shown in Table 3. The recovery rates were in the range of 100±20%, which indicates that measurements were properly performed.

TABLE 3

| | | Calibration curve | Detection | Dilution | Recovery |
|---|---|---|---|---|---|
| | Swiss-Prot | measuring range | limit | factor | rate |
| Protein | accession no. | (ng/mL) | (ng/mL) | (%) | (%) |
| α-enolase | P06733 | 0.625-40 | 0.534 | 5 | 108 |
| Calnexin | P27824 | 12.5-200 | 2.59 | 20 | 81.9 |
| CNDP dipeptidase 2 | Q96KP4 | 6.25-800 | 4.83 | 2 | 94.1 |
| Galectin-1 | P09382 | 1.95-125 | 0.0273 | 10 | 96.1 |
| Galectin-3 | P17931 | 0.313-40 | 0.275 | 10 | 81.0 |
| Lectin mannose-binding 2 | Q12907 | 23.4-3000 | 3.90 | 5 | 83.8 |
| MHC class I antigen A | P18462 | 25-1600 | 20.0 | 20 | 81.2 |
| Triosephosphate isomerase | Q14773 | 12.5-1600 | 0.617 | 5 | 89.7 |

### Example 2

Plasma samples collected from 51 healthy individuals and 15 renal cancer patients were used to measure blood concentrations and compare the blood concentrations between the group of healthy individuals and the group of renal cancer patients. The male-to-female ratio, average age, and age range of each group of healthy individuals and renal cancer patients, the pT classification of renal cancer, presence or absence of distal metastasis, and the position of tumor (right or left kidney) are shown in Table 4.

TABLE 4

| Healthy individuals | |
|---|---|
| Number of samples | 51 |
| Female (%) | 15 (29.4) |
| Male (%) | 36 (70.6) |
| Average age (Age range) | 62.8 (40-82) |
| Renal cancer patients | |
| Number of samples | 15 |
| Female (%) | 3 (20.0) |
| Male (%) | 12 (80.0) |
| Average age (Age range) | 65.1 (39-79) |
| pT classification | |
| pT1 | 9 |
| pT2 | 2 |
| pT3 | 4 |
| Distal metastasis | |
| absence | 11 |
| presence | 4 |
| Position of tumor | |
| Left kidney | 6 |
| Right kidney | 9 |

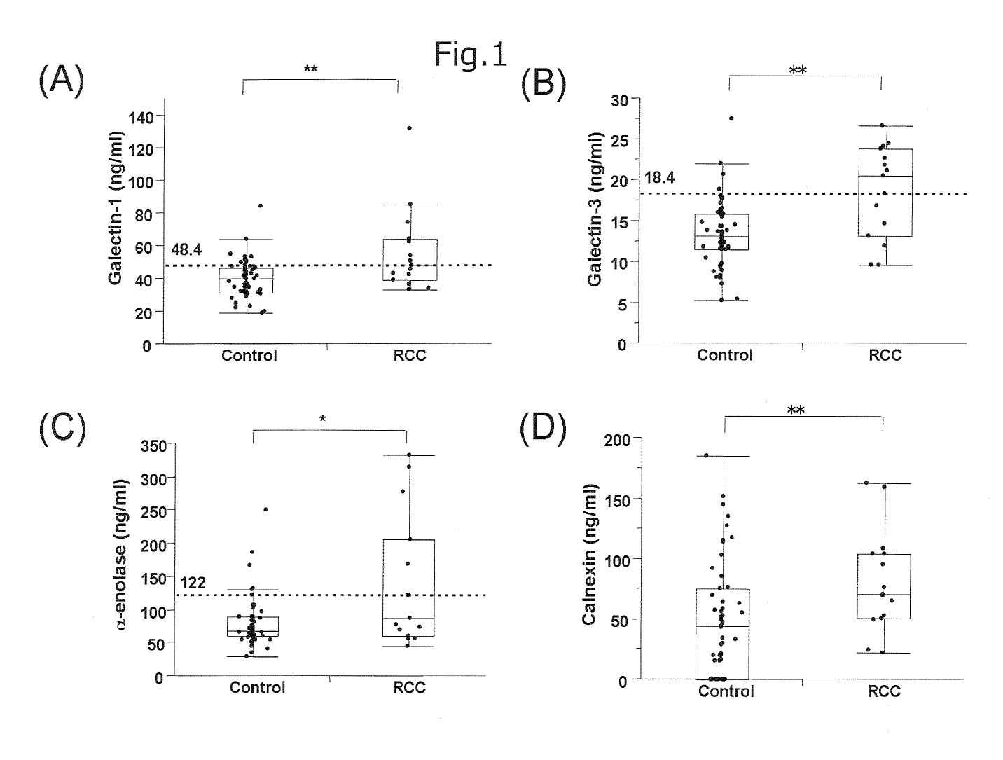

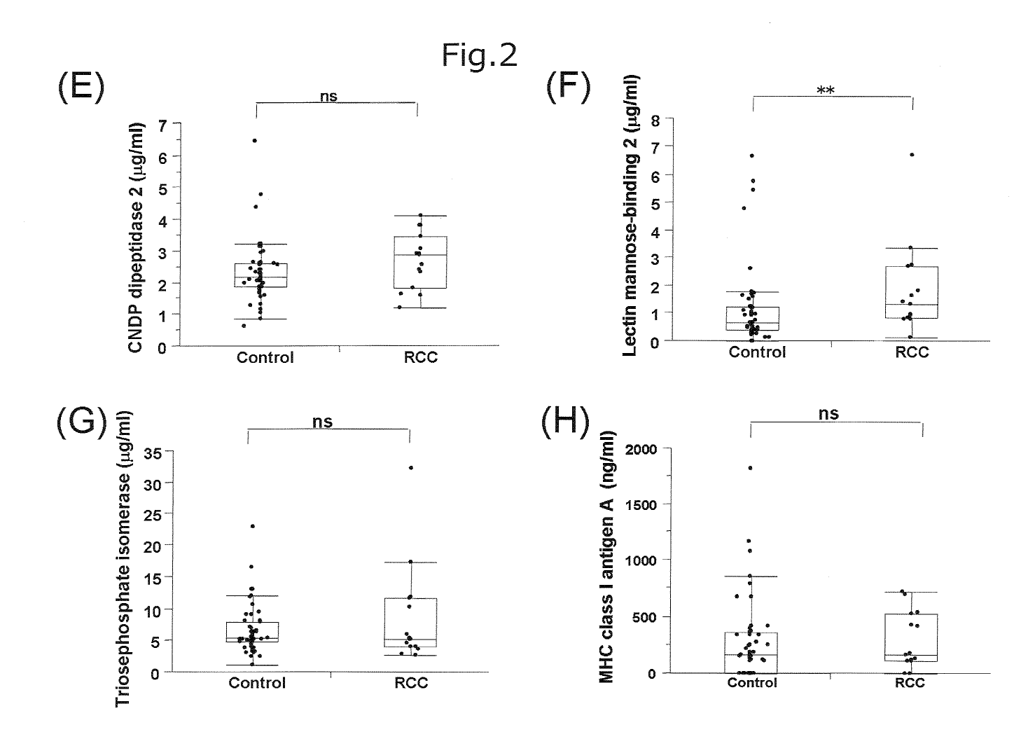

FIGS. 1 and 2 show graphs each representing the comparison of blood concentration of Glaectin-1 (FIG. 1A), Glaectin-3 (FIG. 1B), α-enolase (FIG. 10), Calnexin (FIG. 1D), CNDP dipeptidase 2 (FIG. 2E), Lectin mannose-binding 2 (FIG. 2F), Triosephosphate isomerase (FIG. 2G), or MHC class I antigen A (FIG. 2H) between the group of healthy individuals (Control) and the group of renal cancer patients (RCC). Each box for the Control or RCC group represents the range from the 25th to 75th percentile of concentration distribution of all the samples (interquartile range), horizontal lines above and below the box represent the maximum and minimum values of the samples within 1.5 times the interquartile range from the upper and lower ends of the box, respectively, and a horizontal line in the box represents the median concentration.

The blood concentrations of the above-mentioned eight proteins were statistically compared between the healthy individuals and the renal cancer patients by Mann-Whitney test. As a result, Galectin-1, Galectin-3, α-enolase, Calnexin, and Lectin mannose-binding 2 yielded a p-value of 0.05 or less, from which it has been found that they show significant differences in their blood concentrations between the healthy individuals and the renal cancer patients. However, many of the healthy individuals had blood concentrations of Calnexin and Lectin mannose-binding 2 higher than the third quartile points of the renal cancer patients. From the results, it has been found that Calnexin and Lectin mannose-binding 2 have low specificity and are likely to yield false positive results. Therefore, Calnexin and Lectin mannose-binding 2 are considered to be less useful as markers. On the other hand, CNDP dipeptidase 2, Triosephosphate isomerase, and MHC class I antigen A showed no significant differences in their blood concentrations between the healthy individuals and the renal cancer patients.

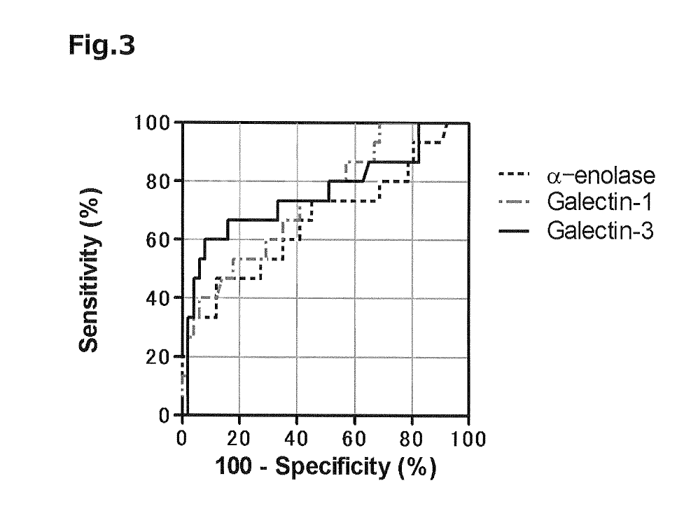

In order to verify the usefulness of Galectin-1, Galeectin-3, and α-enolase as markers, ROC curves were generated using the above-described measurement data (FIG. 3).

Table 5 shows the medians and interquartile ranges of blood concentrations, and the AUCs in relation to Galectin-1, Galectin-3, and α-enolase.

TABLE 5

| | α-enolase | Galectin-1 | Galectin-3 |
|---|---|---|---|
| Protein | (ng/mL) | (ng/mL) | (ng/mL) |
| Healthy individuals (number of samples = 51) | | | |
| Median; Interquartile range | 67.7; 59.6-88.6 | 39.8; 32.1-46.5 | 13.3; 11.5-15.9 |
| Renal cancer patients before surgery | | | |
| (number of samples = 15) | | | |
| Median; Interquartile range | 87.7; 60.7-206 | 48.5; 39.1-64.6 | 20.5; 13.1-23.9 |
| P value * | 0.0493 | 0.0055 | 0.0024 |
| AUC (Area Under the ROC Curve) | 0.6673 | 0.7379 | 0.7601 |
| Renal cancer patients after surgery | | | |
| After 1 week of surgery | | | |
| (number of samples = 14) | | | |
| Median; Interquartile range | 77.6; 57.8-245 | 60.4; 52.1-70.9 | 18.6; 13.2-22.4 |
| P value^{†}: | 0.3575 | 0.1937 | 0.8077 |
| before surgery vs after 1 week of surgery | | | |
| After 2 weeks of surgery | | | |
| (number of samples = 14) | | | |
| Median; Interquartile range | 134; 64.7-315 | 77.2; 61.6-110 | 20.3; 15.1-27.7 |
| P value^{†}: | 0.1937 | 0.0353 | 0.0295 |
| before surgery vs after 2 weeks of surgery | | | |
| After 4 weeks of surgery | | | |
| (number of samples = 13) | | | |
| Median; Interquartile range | 68.2; 53.7-87.6 | 67.1; 54.1-92.2 | 17.6; 14.9-26.0 |
| P value^{†}: | 0.0034 | 0.0942 | 0.8926 |
| before surgery vs after 4 weeks of surgery | | | |
| After 12 weeks of surgery | | | |
| (number of samples = 8) | | | |
| Median; Interquartile range | 62.6; 49.5-80.7 | 69.9; 54.9-81.1 | 22.3; 12.3-24.7 |
| P value^{†}: | 0.0156 | 0.250 | 0.5469 |
| before surgery vs after 12 weeks of surgery | | | |

The ROC curves of all proteins showed satisfactory behavior. The ROC curves of both Galectin-1 and Galectin-3 have an AUC (area under the ROC curve) of larger than 0.7, from which it has been found that Galectin-1 and Galectin-3 deliver particularly excellent performance as markers.

Particularly, as can be seen from FIG. 3, Galectin-3 yields a detection rate (sensitivity) of higher than 60% under the condition that the specificity is 80% or higher (it means that such a threshold value can be set), that is, Galectin-3 has excellent sensitivity under the condition that the false-positive rate is low. Therefore, among the marker proteins according to the present invention, Galectin-3 is most useful as a marker for early detection. Further, Galectin-1 and α-enolase are also useful as markers for early detection because they yield a sensitivity of about 50% under the condition that the specificity is 80%.

It is known that β-enolase and y-enolase exist as enolase family proteins. Therefore, these family proteins were subjected to a cross-reactivity test using the α-enolase ELISA measurement system. As a result, the cross-reactivity of β-enolase was 0.00% and the cross-reactivity of y-enolase was 1.41%. From these results, it has been confirmed that the family proteins other than α-enolase hardly influence the results of α-enolase measurement.

### Example 3

In order to use the proteins as markers with high accuracy, it is necessary to set their threshold values that indicate both high sensitivity and high specificity. Accordingly, the concentration of each of the proteins that maximized Youden's Index was determined using the ROC curve obtained in Example 2 to set each of threshold values. As a result, a Galectin-1 concentration of 48.4 ng/mL, a Galectin-3 concentration of 18.4 ng/mL, and an α-enolase concentration of 122 ng/mL were derived. At the respective concentration values, Galectin-1 showed a sensitivity of 53.3% and a specificity of 82.4%; Galectin-3 showed a sensitivity of 60.0% and a specificity of 92.2%; and α-enolase showed a sensitivity of 46.7% and a specificity of 88.2%.

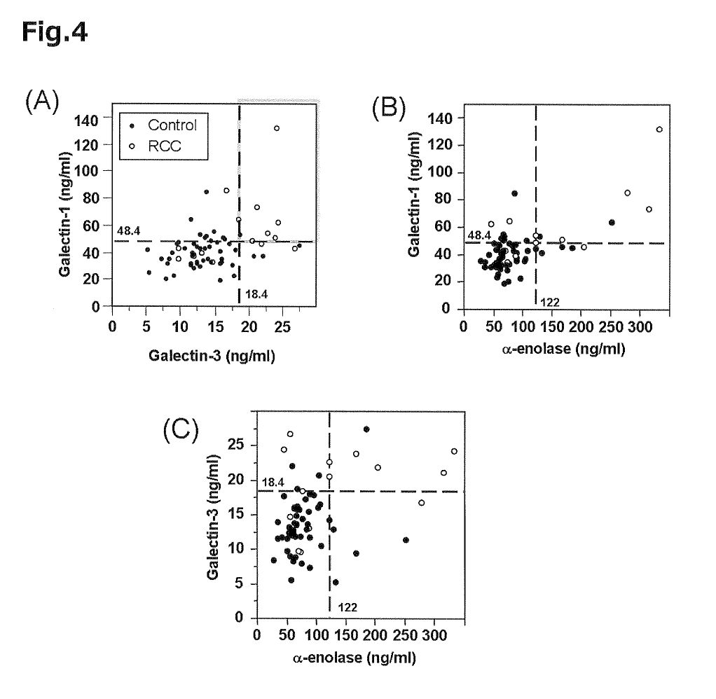

FIGS. 4A to 4C are scatter diagrams each obtained by plotting the concentrations of any two of the three proteins of the renal cancer patients (RCC) and the healthy individuals (Control). In these diagrams, the threshold values set in such a manner as described above are indicated by broken lines. When the case where the concentrations of both proteins exceed the threshold values was regarded as a positive case, a combination of Galectin-1 and Galectin-3 yielded a specificity of 98.0% (50/51) and a sensitivity of 46.7% (7/15) (FIG. 4A); a combination of Galectin-1 and α-enolase yielded a specificity of 96.1% (49/51) and a sensitivity of 40.0% (6/15) (FIG. 4B); and a combination of Galectin-3 and α-enolase yielded a specificity of 98.0% (50/51) and a sensitivity of 40.0% (6/15) (FIG. 40). From these results, it has been found that such combined use of the markers improves specificity (i.e., reduces a false-positive rate) as compared to when the markers are used alone. It is to be noted that when markers are combined by intersection, sensitivity is inevitably reduced. However, the combination of Galectin-1 and Galectin-3 is particularly preferred because it shows a smaller reduction in sensitivity than the other combinations.

### Example 4

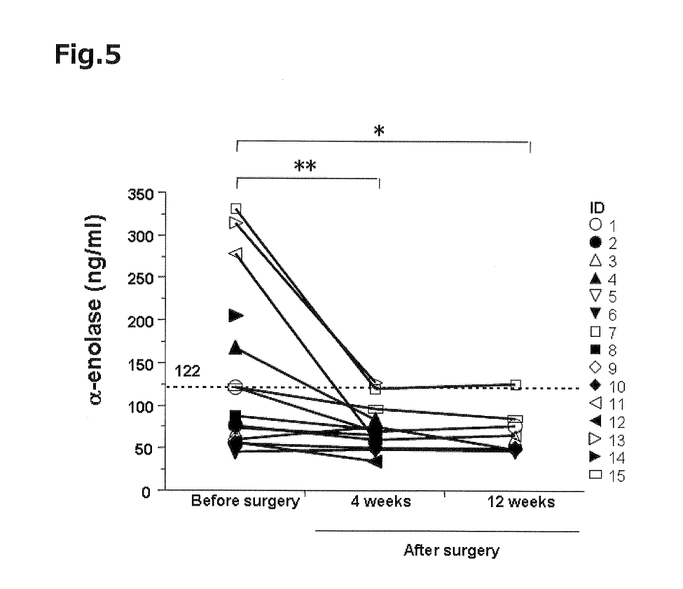

Plasma samples prepared from blood collected from renal cancer patients before surgery, 4 weeks after surgery, and 12 weeks after surgery were used to determine changes in the blood concentrations of the three proteins (Galectin-1, Galectin-3, and α-enolase) before and after surgery. As a result, it was found that many of the patients with a high concentration of blood α-enolase before surgery had a reduced blood concentration after surgery. FIG. 5 shows changes in the blood α-enolase concentration before and after surgery. Five of the seven patients who were (renal-cancer) positive before surgery based on the threshold value determined in Example 3 had α-enolase concentrations reduced to below the said threshold 4 weeks after surgery. One of the remaining two cases was excluded because there was no sample collected 4 weeks after surgery. In the other case, the blood concentration of α-enolase was found to be reduced to a value not equal to or below the threshold value but very close to a value indicating negative response.

From these results, it has been found that the concentration of α-enolase in blood can be used as an indicator of the amount of cancer present in the body. Therefore, α-enolase is considered as a marker usable as an indicator of relapse of renal cancer after treatment, or as an indicator for evaluation of therapeutic effects during treatment.

What is claimed is:

1. A blood marker for renal cancer selected from the group consisting of Galectin-1, Galectin-3, and α-enolase.

2. Galectin-1 as a blood marker for renal cancer for use in an examination performed before diagnostic imaging.

3. Galectin-3 as a blood marker for renal cancer for use in an examination performed before diagnostic imaging.

4. A combination of Galectin-1 and Galectin-3 as a blood marker for renal cancer for use in an examination performed before diagnostic imaging.

5. α-Enolase as a blood marker for renal cancer for use in an examination performed before diagnostic imaging.

6. α-Enolase as a blood marker for renal cancer for use in monitoring during and/or after treatment for renal cancer.

7. A renal cancer marker detection kit comprising an enzyme-labeled α-enolase antibody.

8. A method for analyzing a renal cancer marker in a collected blood sample, the method comprising the step of acquiring a level of a renal cancer marker selected from the group consisting of Galectin-1, Galectin-3, and α-enolase in a collected blood sample derived from an individual, and evaluating the acquired marker level based on whether said acquired marker level is higher or lower than a reference level of the renal cancer marker.
